(19) United States
(12) Patent Application Publication
(10) Pub. No.: US 2011/0207120 A1
(43) Pub. Date: Aug. 25, 2011

(54) WNT1 AS A RENAL DAMAGE BIOMARKER

(75) Inventors: Elisenda Bañon-maneus, Barcelona (ES); Luis Fernando Quintana Porras, Barcelona (ES); Josep Maria Campistol Plana, Barcelona (ES)

(73) Assignee: HOSPITAL CLINIC I PROVINCIAL DE BARCELONA, Barcelona (ES)

(21) Appl. No.: 13/059,036

(22) PCT Filed: Aug. 12, 2009
(86) PCT No.: PCT/EP09/60403

§ 371 Date: May 2, 2011

(30) Foreign Application Priority Data
Aug. 14, 2008 (ES) P200802442

(51) Int. Cl. C12Q 1/68; G01N 33/68; C07K 16/18; C07H 21/00

(52) U.S. Cl. 435/6.1; 436/501; 435/7.1; 530/387.9; 536/23.1

(57) ABSTRACT

The present invention comprises the use of WNT1 as a biomarker in the monitoring, prognosis and/or in the diagnosis of chronic nephropathies. It provides in vitro prognostic and diagnostic methods and kits for its implementation. The present invention also comprises the use of WNT1 in screening active ingredients for the manufacture of a drug for therapies for chronic nephropathies and a method for conducting said screening.

### FIELD OF THE INVENTION

The present invention is framed in the field of clinical tools for the diagnosis and monitoring of renal function in patients with chronic renal failure (CRF), especially those who have undergone transplant therapy.

### BACKGROUND OF THE INVENTION

Chronic renal failure (CRF) consists of a slow and progressive loss of renal function, characterized by a low Glomerular Filtration Rate (GFR). When renal failure is very severe (End Stage Renal Disease, ESRD), replacement therapy is required which can consist of either dialysis or renal transplantation.

Renal transplantation therapy is a successful alternative that can prolong the patient's life up to more than 15 years in some cases. However, there are multiple risks and complications. Despite the fact that tissue compatibility tests have been improved in recent years, it is necessary to develop in parallel continuous immunodepressive therapy for the purpose of preventing acute or chronic rejections which may lead to renal function failure of the transplanted organ. After transplantation, high levels of serum creatinine are indicative of failure in the function of the transplanted kidney. However, the creatinine method is neither sensitive nor specific. Accordingly, the development of monitoring tools for monitoring renal function of transplanted kidneys and of evaluation tools for evaluating graft survival has become a clinical necessity. Chronic allograft nephropathy (CAN), characterized by interstitial fibrosis and tubular atrophy, is the leading cause of transplanted organ loss. CAN has a multifactorial etiology in which both immunological factors (allograft rejection) and non-immunological factors, especially nephrotoxicity due to calcineurin inhibitors, are involved. Today there are a number of monitoring tools in the form of a diagnostic kit which allows detecting rejection of the transplanted organ based on the specific activity of the patient's immune system. For example, application WO 2004074815 A1 teaches a method for evaluating functional failure or rejection risk of a transplanted organ from a tissue biopsy or blood sample which consists of determining the level of expression of one or more genes encoding for proteins associated with inflammation. Similarly, patent application WO 2006099421 A1 describes methods for evaluating the progress of the transplanted organ, identifying the presence of functional damage, such as for example chronic allograft nephropathy, and identifying the severity and class of acute rejection (AR). The methods described therein comprise the detection, at the protein or nucleic acid level in blood or biopsy, of at least one gene specified in Tables 1 and 2. Table 2 specifies the 30 predictive genes for said methods using blood or tissue from a renal biopsy. Remarkably, all these genes are associated with immune system activity. They correspond either with cytokine- or chemokine-induced genes or with genes forming part of the MHC complex, genes of the complement or immunoglobulins. The 479 genes in Table 3 represent as a whole an example of “transplant chip” including both the genes of Tables 1 and 2 as other genes characteristic of allograft nephropathies (AR, CAN). Also included among them are control genes and modulator genes of the normal function of the immune system, identified in a review of the literature.

The type of clinical tools mentioned above require invasive methods for obtaining samples, for example, biopsies which involve a considerable associated morbidity in addition to involving a considerable economic cost. An alternative to blood or tissue samples is a urine sample; however, today there are no reliable clinical tools for diagnosis the status of the transplanted organ from urine samples. In the case of a transplanted kidney, sensitive techniques have been developed for detecting the presence in urine of proteins associated with the inflammation process. The work of Dr. Nickerson's team in Canada used proteomic technology to detect urinary proteins associated with AR (Schaub et al., J Am Soc Nephrol. 2004 January; 15(1):219-27). Some companies interested in biomedical technology are investing their efforts in this direction also (WO 07121922 A2 and WO 07104537 A2, Am J Transplant. 2005 October; 5(10):2479-88; CA 2473814 A1). These applications and studies contemplate the possibility of detecting biomarkers relating to the function of the immune system in urine, however there are no reliable commercial clinical methods using this technology. This is because even though proteomic technology has the potential of clarifying complex aspects of pathophysiological processes and of disclosing new biomarkers, the current state of the urinary proteome of renal transplantation pathologies is still far from achieving such objectives (Schaub et al., Contrib Nephrol. 2008; 160:65-75).

As mentioned above, the characteristic signs of CAN are interstitial fibrosis and tubular atrophy. It is known that the etiology of CAN partly lies on rejection of the transplant. However there are no direct tools available which detect early damage in the grafted tissue, especially by means of non-invasive techniques such as urinalysis. One of the problems derived from using biomarkers relating to immune system activity is that they do not enable distinguishing between acute infection and rejection. Furthermore, these methods do not directly reflect the renal function status and cannot be applied in evaluating patients who preserve their own kidney.

Therefore, in daily clinical practice the main problem for professionals is that they do not have sufficient non-invasive diagnostic tools showing the existence of renal damage. As it has been shown, there are tools which detect rejection as a function of immune system activity, but with invasive techniques.

Surprisingly, the inventors of the present application have identified a specific fibrosis marker in urinalysis. The analysis of kidney transplant patient samples by means of the 2D-DIGE proteomic technique has shown the distinctive presence of the protein WNT1 in the urine of those patients suffering chronic allograft nephropathy. The WNT1 protein is not expressed in the adult kidney, but during development it induces metanephric mesenchyme to differentiate into tubular and glomerular epithelium (Herzlinger et al., 1994; Dev. Biol. 166:815-818) and it could be involved in fibrosis and tissue atrophy processes in the lung (Königshoff et al., PLoS ONE. 2008 May 14; 3(5):e2142).

The present invention therefore provides a new non-invasive clinical tool which allows a direct measurement of tissular damage in the kidney at an early stage through the analysis of a patient's urinary sample.

### BRIEF DESCRIPTION OF THE DRAWINGS

FIG. 1 shows the detail of detecting the wnt-1 protein by Western-blot. Two random samples of each group of the patients complying with the study inclusion criteria were chosen to detect wnt-1. Figure a) corresponds to the detail of two renal transplant patients without CAN (CAN 0), b) corresponds to the detail of two renal transplant patients with incipient CAN (CAN I) and c) corresponds to the detail of two renal transplant patients with advanced CAN (CAN II-III). CAN: Chronic allograft nephropathy.

FIG. 2 shows the images provided by DeCyder® image analysis software (GE Healthcare). The area delimited by the line corresponds to the point of the protein identified as wnt-1. It can be observed that the height of the area increases as the severity of the CAN increases, this increase of the area corresponds with an increase of the amount of protein in urine. Figure a) corresponds to the detail of renal transplant patients without CAN (CAN 0), b) corresponds to the detail of renal transplant patients with incipient CAN (CAN I) and c) corresponds to the detail of renal transplant patients with advanced CAN (CAN II-III). CAN: Chronic allograft nephropathy.

### Definitions

In the context of the present invention, the term WNT1 relates to, unless expressly specified, otherwise any of the biological forms of the gene wingless-related MMTV integration site 1 (gene locus 12q12-q13 in Homo sapiens) and combinations thereof. Said biological forms comprise but are not limited to DNA, variants and mutations thereof, control regions thereof such as regulators, modulators, promoters and enhancers; cDNA and constructs comprising it; RNA in any of its versions, including mRNA and the protein, the post-translational modifications, mutations and versions thereof and fragments thereof. Biomarker is also understood as any biological molecule which is distinctive of a physiopathological process. In the case of the present invention, said process corresponds with interstitial fibrosis and the tubular atrophy, which are characteristic of renal function impairment.

### BRIEF DESCRIPTION OF THE INVENTION

A first aspect of the present invention is the use of WNT1 as a biomarker in the prognosis of renal function impairment and/or in the diagnosis of nephropathies associated with said impairment. In preferred embodiments, the present invention comprises this use in kidney transplant patients.

Another aspect of the present invention is a method for the prognosis of renal function impairment and/or for the diagnosis of nephropathies associated with said impairment, comprising the determination of the presence or absence of the biomarker WNT1, or a fragment thereof, in a biological sample isolated from a patient. In a preferred embodiment, the biological sample used is urine, blood, serum or tissue biopsy and it comprises the determination of the presence or absence of the protein, RNA or DNA of WNT1 or a fragment thereof.

In preferred embodiments, the method of the present invention comprises a biological sample isolated from a renal transplant patient. In still more preferred embodiments, the method of the present invention comprises the quantification of WNT1 in the samples.

A third aspect of the present invention comprises a method for the in vitro diagnosis of chronic allograft nephropathy, said method comprising:
a) The quantification of the WNT1 or a fragment thereof in a biological sample isolated from a patient.
b) The comparison of the amount of WNT1 in the sample of step a) with the amount of WNT1 in samples isolated from healthy individuals.

In this method, the presence or the relative increase of the amount of WNT1 are indicative of renal function impairment.

In very preferred embodiments, this method is performed using patient urine samples. In other embodiments, blood, serum or biopsy tissue samples are used in the method.

An additional aspect of the present invention is a kit for the monitoring, prognosis and/or diagnosis of renal function impairment and nephropathies associated with said impairment comprising at least one molecule or composition able to bind to and recognize a sequence corresponding with any of the biological forms of WNT1 and selected from SEQ ID No: 1, SEQ ID No: 2, SEQ ID No: 3, or a fragment thereof; said molecule is optionally labeled to facilitate detection thereof.

Another additional aspect of the present invention is a kit for the monitoring, prognosis and/or diagnosis of the renal function impairment or nephropathies associated with said impairment comprising the biomarker WNT1 or a fragment thereof.

A particular embodiment of the present invention comprises the use of said kit in screening active ingredients for the manufacture or the development of drugs intended for the treatment of diseases resulting from fibrogenesis processes.

An aspect of the present invention is also a method for screening active ingredients for the manufacture or the development of a drug comprising a binding assay of said active ingredient to WNT1.

In preferred embodiments, the kits of the present invention are aimed at the monitoring, prognosis and/or the diagnosis or at screening active ingredients or the manufacture of drugs for therapy for the allograft nephropathies associated with renal function impairment.

A final aspect of the present invention is the use of WNT1 or a fragment thereof in screening active ingredients for the manufacture of a drug for the treatment of nephropathies. In a preferred embodiment, said nephropathies are chronic allograft nephropathies.

### DETAILED DESCRIPTION OF THE INVENTION

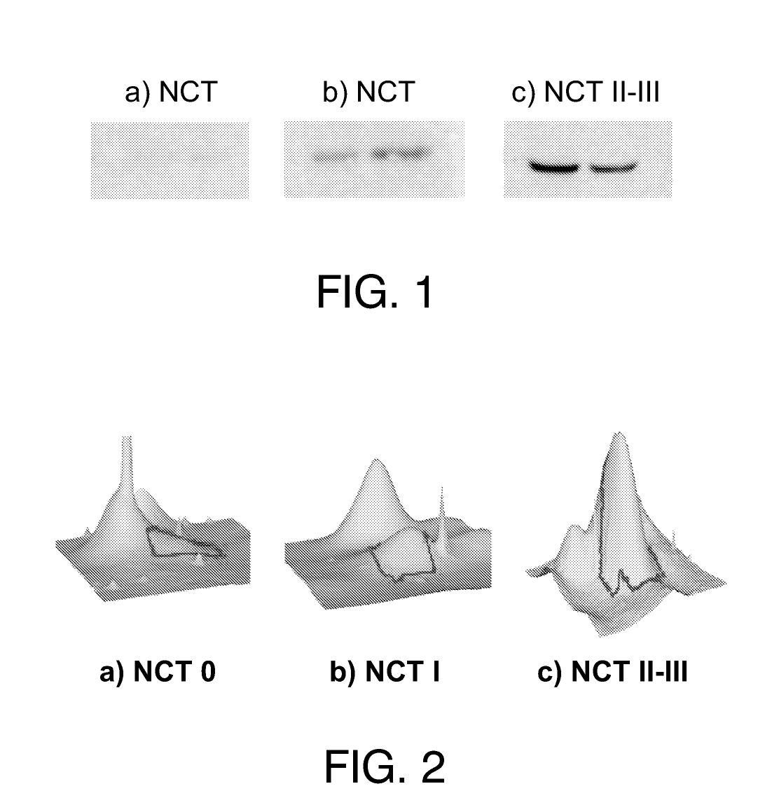

The present invention is based on the unexpected observation made by the inventors of the presence of the WNT1 protein in the urine of patients with CAN (FIG. 1). The absence of WNT1 in the urine of patients who do not suffer chronic allograft nephropathy or renal transplant patients without chronic allograft nephropathy or general transplant population with normal renal function makes WNT1 a biomarker with a high diagnostic and predictive value for said patients. The inventors attribute the expression of WNT1 to regenerative processes which, when failing in the adult kidney, lead to interstitial fibrosis, tubular atrophy and the formation of the sclerotic lesions observed in biopsies. Such regenerative processes would begin, in the transplanted kidney, immediately with the first lesions caused by acute rejection and/or other injurious insults. In correspondence with the proliferation of lymphocytes, the thickening of the intimal layer and the disruption of the elastic layer occur. The inventors have thus observed that unlike other markers used in the technical field, with respect to the presence or absence of WNT1 in patient samples, may lead to a direct measurement of the damage and atrophy of the structures carrying out the renal function. The inventors speculate that WNT1 could be involved in the formation of neo-media and neo-intima observed in very early stages of chronic rejection.

Since the expression of WNT1 cannot be a consequence of the surgical mechanics of the transplant, but rather it corresponds with the intrinsic physiopathology of the kidney, these findings can be extrapolated and applied also to patients suffering impairment of their natural kidney.

It would be very desirable in daily clinical practice to have an analytical tool that could be obtained from a sample isolated from the patient the collection of which did not negatively influence the patient's quality of life, involved no morbidity for the patient or a high economic cost, such as, for example, urine samples. In accordance with these desirable improvements, a first aspect of the present invention is the use of WNT1 as a biomarker in the prognosis of renal function impairment and/or in the diagnosis of nephropathies associated with said impairment.

According to the present invention, the nephropathies associated with renal function impairment comprise diabetic nephropathy, nephroangiosclerosis, IgA nephropathy, membranous nephropathy, focal segmental glomerulosclerosis, lupus nephritis (associated with systemic lupus erythematosus), ANCA-positive pauci-immune crescentic glomerulonephritis (associated with anti-neutrophil cytoplasmic antibodies in plasma) and chronic allograft nephropathy, among others. Some of these nephropathies can occur both in transplant and non-transplant patients.

Some of the disorders presenting with interstitial fibrosis and tubular atrophy furthermore comprise infectious diseases such as AIDS or chronic autoimmune diseases, such systemic lupus erythematosus mentioned above.

It would also be desirable in daily clinical practice to have an analytical tool which, being able to indicate an early stage of CAN, was not a measurement of immune system activity in order to be able to thus distinguish between an acute infection and a graft rejection. According to this improvement, in some embodiments the present invention comprises the use of WNT1 as a biomarker in the prognosis of renal graft function impairment and/or in the diagnosis of nephropathies associated with said impairment in kidney transplant patients.

According to the present invention, the nephropathies associated with renal function impairment comprise, in addition to those already mentioned, any of the disorders presenting with interstitial fibrosis and tubular atrophy.

For the purpose of aiding to assess, evaluate and determine the specific symptoms of renal function impairment, another aspect of the present invention comprises a method for the monitoring, the prognosis of renal function impairment and/or for the diagnosis of nephropathies associated with said impairment comprising the determination of the presence or absence of WNT1, or a fragment thereof, in a biological sample isolated from a patient.

When the method is applied to the prognosis, the invention contemplates technical assistance in the assessments about the risk that said patient suffers one of the diseases or disorders mentioned for this invention by means of providing specific data about the presence or absence of any biological form of WNT1.

In a preferred embodiment, the biological sample is urine, blood, serum or tissue biopsy and it comprises the determination of the presence or absence of the protein, RNA or DNA of WNT1 or a fragment thereof.

The possible embodiments of the method of the present invention comprise:
a) The collection of samples from the patient. The samples will be used immediately or suitably preserved, depending on their nature.

For example, the samples can be processed immediately or they can be vacuum packaged or frozen at −80° C. until their analysis to prevent degradation of the biological forms of WNT1. The treatment of the samples after their collection is in no case limiting for the object of the present invention and will be done according to the best protocol known by a person skilled in the art at the time of carrying out the method of the present invention.
b) The isolation of the fraction from the sample and the detection therein of the chosen biological form of WNT1.

If, for example, the presence or absence of the protein WNT1 is chosen to be analyzed, the sample will be centrifuged and subjected to protein concentration protocols. If the sample is a blood sample, it will be necessary to eliminate the cell fraction prior to said concentration. In the case of a renal tissue biopsy, the specific treatment described in immunohistochemistry protocols or any other technique for detecting proteins in tissue known in the field of the art will be followed. Commercial specific anti-WNT1 antibodies will be used for that purpose. These antibodies can be diluted in solutions for the treatment of said fractions of the samples together with other reagents or they can be fixed to solid supports to facilitating the binding of the protein to said support and the subsequent development thereof in, for example, an ELISA-type or affinity immunochromatography-type assay. If, however, the gene expression of WNT1 is chosen to be analyzed, suitable mRNA extraction protocols which will include the addition to the latter of a potent RNase inhibitor will be chosen. Said protocols are known in the field of the art and may vary according to the nature of the sample. For example, in the case of biopsy, it will require the homogenization of the tissue and a RT-PCR protocol which can be quantitative and for which suitable primers will be required which the person skilled in the art will choose according to his best knowledge.

C) Optionally, the quantification of the chosen biological form of WNT1 in the fraction isolated in step b).

Although the mere presence of mRNA or WNT1 protein in the sample is indicative of renal function impairment, a tool allowing quantification would be desirable because such quantification may serve to determine the degree of renal function loss. According to this, the present invention comprises the quantification of WNT1 as a clinical tool in the evaluation of symptoms for the most appropriate diagnosis in each patient.

In clinical nephrology, the professional in charge of diagnosing patients who suffer renal failure lack sufficient tools providing objective technical data about the structural degradation of the kidney. The present invention offers the possibility of applying the detection and quantification of WNT1 to improve this clinical deficiency. Therefore, one embodiment of the present invention comprises a method for the in vitro diagnosis of chronic allograft nephropathy comprising:
c) Quantification of the WNT1 or a fragment thereof in a biological sample isolated from a patient.
d) Comparison of the amount of WNT1 in the sample of step a) with the amount of WNT1 in samples isolated from healthy individuals,
   wherein the presence or the relative increase of the amount of WNT1 are indicative of renal function impairment.

An example of this quantification can be seen in FIG. 2.

A particularly annoying aspect for the patient is the reduction of his quality of life since he must systematically undergo tests to obtain renal biopsies in order to provide objective technical data about the degree of his kidney tissue degradation. The present invention represents an improvement in this sense because it allows obtaining said data from a urine sample. In a very preferred embodiment, the method of this invention for the in vitro diagnosis of chronic allograft nephropathy comprises the use of urine samples obtained from the patient.

The presence of WNT1 in samples from patients can be quickly and specifically detected by means of the use of a set of selected reagents. To that end, in another aspect, the present invention comprises a set of reagents or kit for obtaining molecular data aiding in the monitoring, prognosis and/or diagnosis of renal function impairment or nephropathies associated with said impairment. This set of reagents comprises at least one molecule or composition able to bind to and recognize one of the biological forms of WNT1, i.e., a sequence selected from SEQ ID No: 1, SEQ ID No: 2, SEQ ID No: 3, or a fragment thereof. Said molecule can optionally be labeled for its detection.

These reagents traditionally comprise, in the case of nucleic acids, artificially synthesized sequence fragments in which radioactive molecules or molecules able to print radiophotographic film have been included. In an equivalent manner, the reagents used in the detection of proteins are traditionally antibodies which can be labeled with radioactive, fluorescent or luminescent molecules. The present invention contemplates fixing these antibodies to a solid support to create a kit that can be used in an ELISA-type assay.

The kit described above is particularly useful in the identification of individuals at risk of developing a nephropathy. To that end, said kit can serve as a means for detecting said individuals and developing a strategy of preventive measures or intervention therapies, working before the occurrence of irreversible damage or before the development of the disease. Particularly, this kit serves as an aid to clinical staff in the follow-up and monitoring of the progression of the disease, as well as of the success or ineffectiveness of the chosen therapy.

Given that interstitial fibrosis and tubular atrophy cause chronic renal failure, it would be convenient to block the agent which causes them. In this sense, another embodiment of the present invention provides an alternative kit for the one described above comprising among its reagents at least one biological form of WNT1. This alternative kit is useful in the development of assays for screening, for example, molecules able to promote or inhibit gene expression for example by means of the binding to the promoter of the WNT1 gene; molecules able to prevent the translation or transcription of the gene or block the secretion or the binding of the WTN1 protein to its receptor. This kit is furthermore useful in the manufacture of new drugs having WNT1 as a therapeutic target. Likewise, this alternative kit can benefit both the clinician and the patient providing a means for the development of assays for the early detection of renal damage or the progress of a transplanted kidney. This kit thus comprises a matrix or solid support to which any of the biological forms of WNT1 and alternatively other known biomarkers would bind. Therefore, another aspect of the present invention is a method for screening active ingredients for the manufacture or the development of a drug comprising a binding assay of said active ingredient to WNT1.

Thanks to the technology provided by the present invention, a patient, for example a renal transplantation patient, can be incorporated to a program for the follow-up of the functional progress of his or her transplanted kidney which would allow an early intervention in the event of rejection or dysfunction. According to this, preferred embodiments of the present invention comprise a kit aimed specifically at the prognosis and/or the diagnosis of allograft nephropathies as well as the monitoring of the transplanted organ.

Therefore, a final aspect of the present invention comprises the therapeutic usefulness in the event that a renal patient develops a chronic allograft nephropathy. According to the present invention, said therapeutic usefulness comprises the use of WNT1 in screening for active ingredients and/or in the manufacture and selection of a drug for the treatment or the prevention of a nephropathy. An embodiment of the present invention very preferably comprises said use when the nephropathy is chronic allograft nephropathy.

### EXAMPLES

### Example 1

### Detection of wnt-1 in the Urine of Transplanted Patients Affected or not by CAN

### 1.1 Patients

The second urine of the morning of renal transplant patients was collected at different post-transplantation times. The inclusion criteria were: 1) male gender, 2) stable renal function, 3) post-transplantation time above 6 months, 4) normal sediment and no hematuria, 5) immunosuppressant treatment with Tac+MMF±Pd and 6) recent renal biopsy without signs of acute rejection and the evaluation of chronic lesions performed according to the Banff classification.

The samples were collected from 8 transplant patients with CAN 0, 8 transplant patients with CAN I, 5 transplant patients with CAN II and 3 transplant patients with CAN III. The patients were grouped in three groups: CAN 0 (n=8), CAN I (n=8) and CAN II-III (n=8).

The study was approved and conducted according to the ethics committee of Hospital Clinic de Barcelona. Informed consent was obtained from all the patients.

### 1.2 Preparation of Human Urine Samples

The absence of infection and of hematuria is confirmed by means of test strips (Combur-Test, Roche). 100 ml were collected from the second morning urine from patients with protease inhibitors (Complete Mini and Pefabloc; Roche). The urine was filtered with Whatman 3 mm paper (Whatman, Maidstone, UK) to eliminate the possible solutes from the urine and subsequently centrifuged for 5 minutes at 1,000 g. The supernatant was kept at −80° C. in 40 ml aliquots until its use.

### 1.3 Protein Precipitate

The proteins of the urine are precipitated with TCA (Fluke) at a final concentration of 10%. The protein precipitate was washed twice with acetone at −20° C., the precipitate is subsequently left to dry at 4° C., then it was dissolved in resuspension buffer containing 7 M urea (GE Healthcare), 2 M thiourea (GE Healthcare), 4% CHAPS (GE Healthcare), 0.1% DTT (Sigma), and 0.2% ampholytes with a 4-7 pH range (GE Healthcare). The pH of the samples was brought to pH 8-8.5 with 1 M NaOH to optimize labeling with the fluorochromes of the DIGE assay. The protein concentration was determined with the RcDc Kit (BioRad, according to the protocol of the commercial firm). 30 μl aliquots were taken and stored at −80° C. until their use.

### 1.4. Preparative Gels

### Isoelectrofocusing

24 cm polyacrylamide gel strips were passively rehydrated with a linear pH gradient from 4 to 7 (IPG strips, GE Healthcare) with 450 μl of rehydration buffer containing 2% (w/v) CHAPS (GE Healthcare), 7 M urea (GE Healthcare), 2 M thiourea (GE Healthcare), 0.5% (v/v) ampholytes with a pH range of 4-7 (GE Healthcare), 2 mg/ml dithiothreitol (Sigma) and a trace of bromophenol blue (GE Healthcare). 250 μg of protein were loaded by means of the cup-loading technique (GE Healthcare). The IPG strips were isoelectrofocused at 20° C. in the Ettan IPGphor (GE Healthcare) using the isofocusing program specified in Table 1. Immediately after isoelectrofocusing, the strips are frozen at −80° C. until second-dimension SDS-PAGE is performed.

TABLE 1
Isofocusing Conditions

| | Voltage | Duration | Volts- | Type of |
|---|---|---|---|---|
| Pass | (V) | (h:min) | hour | gradient |
| 1 | 0 | 6:00 | — | — |
| 2 | 50 | 6:00 | — | Step-n-hold |
| 3 | 500 | 1:00 | — | Gradient |
| 4 | 1000 | 1:00 | — | Gradient |
| 5 | 4000 | 1:00 | — | Gradient |
| 6 | 8000 | 1:00 | — | Gradient |
| 7 | 8000 | — | 80,000 | Step-n-hold |

### Second-Dimension: SDS-PAGE

Prior to second-dimension separation, to eliminate the bisulfite bridges the proteins were incubated for 15 minutes at room temperature in equilibration buffer with SDS (50 mM Tris-Cl pH 8.8 (GE Healthcare), 6 M urea (GE Healthcare), 30% (v/v) glycerol (GE Healthcare), 2% (w/v) SDS (Fluka), a trace of bromophenol blue (GE Healthcare) 0.5% (w/v) 1-4 dithiothreitol (DTT) (GE Healthcare)). The IPG strips are subsequently incubated for 15 minutes with the equilibration buffer with iodoacetamide (the buffer is exactly the same as the previous one but with 2.5% iodoacetamide (GE Healthcare) instead of DTT. Buffer solution II is identical to buffer solution I with the exception that it has iodoacetamide rather than DTT. The proteins were separated in the second dimension at 20° C. in 12.5% polyacrylamide gels at 2 W per gel in the Ettan DALT system (GE Healthcare) until the bromophenol blue front eluted (10-14 hours).

### Silver Staining

The separated proteins were viewed using conventional silver staining. Briefly, the proteins are fixed in the gel with the fixing solution (40% ethanol (Merck) and 10% acetic acid (Panreac)) for 30 minutes; the gel was sensitized with the sensitization solution (30% ethanol, 0.2% w/v Na₂S₂O₃ (Amersham Biosciences) and 6.8% w/v sodium acetate (Amersham Biosciences) for 30 minutes. After performing three 5-minute washes with mQ water, the gels were impregnated with a 2.5% w/v silver nitrate solution (Fluka) for 20 minutes. They were subsequently washed twice for 1 min with mQ water. The developing solution (2.5% sodium bicarbonate (Fluka) and 0.4 mL/L formaldehyde (Sigma)) showed the spots. The reaction was stopped by substituting the developing solution with a 1.46% w/v EDTA-Na₂.2H₂O solution (Fluka) for 10 minutes. Finally, three washes were performed with deionized water for 5 minutes each and they were scanned with Molecular Imager® GS-800™ Calibrated Densitometer (Bio-Rad).

1.5 DIGE Two-Dimensional Analysis of the Urinary Proteome in Patients with CAN

### Fluorescent Labeling

Six gels were prepared with the DIGE technique; in each gel the proteome of the total of two patients from one group is compared with the total of two patients from another group, see Table 2. Each sample is labeled with each of the DIGE fluorochromes, Cy2, Cy3, or Cy 5 (GE Healthcare). The proteins were labeled by means of incubation at 4° C. and in the dark with the assigned fluorochrome at a final concentration of 8 pmol fluorochrome per μg protein). The reaction was stopped with 25 mole of lysine per mole of fluorochrome. The samples corresponding to the different groups to be analyzed are labeled with fluorochromes Cy3 and Cy5 for the purpose of analyzing the expression changes according to the stage of the disease. Fluorochrome Cy2 is reserved for labeling intergel control. It is made by mixing identical ratios of all the assay samples. In each gel 50 μg of this intergel control were loaded in each gel with two aims. First, since the intergel control contains all the proteins both of the controls and of the experimental conditions, it produces a reference pattern to compare the patterns of both the analytical and the preparative gels. Second, the intensity of the spots stained with Cy2 serves to compare the intensities of the control and experimental conditions. Before loading the gels, the samples stained with the three fluorochromes were mixed as indicated in Table 2.

TABLE 2

| | Cy 2 | Cy 3 | Cy 5 |
|---|---|---|---|
| Gel 1 | Standard | CAN 0 a | CAN II-III c |
| Gel 2 | Standard | CAN 0 b | CAN II-III d |
| Gel 3 | Standard | CAN I a | CAN 0 c |
| Gel 4 | Standard | CAN I b | CAN 0 d |
| Gel 5 | Standard | CAN II-III a | CAN I c |
| Gel 6 | Standard | CAN II-III b | CAN I d |

Labeling of the totals and mixtures of the 6 gels prepared. CAN 0 a,b,c,d represent the 4 totals of 2 patients/total of renal transplant patients without CAN; CAN I a,b,c,d represent 4 totals of 2 patients/total of renal transplant patients with incipient CAN; CAN II-III represent 4 totals of 2 patients/total of renal transplant patients with advanced CAN. CAN: Chronic allograft nephropathy

### Isoelectrofocusing and SDS-PAGE

Isoelectrofocusing and second-dimension were performed as previously described but all the processes were performed in the dark.

### Image Analysis

As soon as the second-dimension ended, the gels were washed with distilled water and were scanned using the DIGE-enabled Typhoon Scanner (GE Healthcare). The proteins were viewed with the Typhoon Variable Mode Imager (GE Healthcare). The DeCyder Differential In-gel Analysis software (GE Healthcare) was used to analyze the intensity of the spots. The spots of the different gels were aligned using the interassay pattern labeled with Cy2. Specifically, the expression was analyzed for each of the gels in parallel using the DIA module of the DeCyder program using an initial value of 1000 spots present. The DIA analysis was used for the direct comparison of intensities of specific spots between different samples of one and the same gel. In this case, the intensities of the proteins which were compared are of the urinary proteomes of the groups with CAN I, CAN II-III and CAN 0. These DIA analyses were subsequently analyzed with the BVA module of the DeCyder, which allows globally analyzing the expression ratios between the three conditions.

### Identification of the Differential Proteins: Peptide Mass Fingerprinting (PMF) by Means of MALDI-TOF-MS

### In-Gel Digestion

The proteins of interest were excised with the aid of a manual spot picker 1.5 mm in diameter (Gel Company). The proteins were digested with trypsin (Sequencing grade modified, Promega) in the Investigator ProGest robot (Genomic Solutions). Briefly, the excised spots were washed sequentially with ammonium bicarbonate and acetonitrile. After incubation with 10 mM DTT for 30 minutes to reduce the proteins and another incubation with 55 mM iodoacetamide for 30 minutes, the proteins were subjected to sequential buffer and acetonitrile washes. The proteins were digested overnight at 37° C. with 0.27 nmol of trypsin. The peptides obtained from tryptic digestion were extracted from the gel with 10% formic acid and acetonitrile, the extracts were pooled and dried in a vacuum centrifuge.

### Acquisition of Spectra

The proteins excised from the two-dimensional gels were analyzed by means of ESI-MS-MS (Q-TOF Global, Micromass-Waters). The peptides derived from tryptic digestion were analyzed by means of liquid chromatography coupled to mass spectrometry (CapLC-nano-ESI-Q-TOF) (CapLC, Micromass-Waters). In this case, the samples were resuspended in 15 μL of 1% formic acid and 4 μL were injected in the chromatograph to perform reverse-phase separation with C₁₈ (inner diameter of 75 μm and 15 cm in length, PepMap column, LC Packings). The eluted peptides were ionized by means of nano needles (PicoTip™, New Objective). A voltage of 1800-2200 V was applied to the capillary along with a cone voltage of 80 V. The collision in the CID (collision-induced dissociation) is 20-35 eV, the collision gas used is argon. The data generated have PKL format, which allow being subjected to a database search using search tools such as MASCOT or NCBI-Entrez.

1.6. Western Blot of wnt-1

12% acrylamide minigels (Miniprotean, BioRad) 1.55 mm thick were prepared. 25 μg of the urine protein extracts from patients with different degrees of CAN were loaded and were run for 10 minutes at 60V and subsequently at 100V. As soon as the bromophenol blue front eluted, the proteins were transferred to a nitrocellulose membrane (Protan 45 μm in diameter) by means of trans-blot semidry (BioRad) for 30 minutes at 10V.

The membrane was subsequently blocked with a 4% skimmed milk powder solution in PBS for 90 minutes at room temperature. The incubation of the primary antibody (human wnt-1 obtained in rabbit, Rockland) was subsequently performed with a 1:500 dilution in a 1% skimmed milk powder solution overnight (10 hours) at 4° C. and under gentle stirring. After three 10-minute washes, each with a 1% skimmed milk powder solution, the incubation with the secondary antibody (anti-rabbit, SIGMA) was performed with a 1:2000 dilution in a 1% skimmed milk powder solution. After two 10-minute washes, each with a 1% skimmed milk powder solution in PBS, a final wash in PBS was performed. The ECL system (GE Healthcare) was used for the development thereof. The images were subsequently obtained in the LARS image acquisition system.

The result of the identification of the wnt-1 in two patients with CAN 0, CAN I and CAN II-III can be observed in FIG. 1.

### Prophetic Example 2

### ELISA Assay for the Quantification of wnt-1 Present in Urine

To cover the ELISA plate with the wnt-1 antibody, the suitable dilution of the antibody (Roackland) is left to incubate overnight at 4° C. The wells are washed with ddH2O, and the plate is washed twice with PBS-Triton. The plate is blocked with 1% BSA/PBS for 30-60 minutes at room temperature. 100 μl of the standards (known Bionova wnt-1 protein dilutions) and 100 μl of the (perform if dilutions thereof are necessary). It is incubated for an hour at 4° C. The sample is removed and incubated for one hour with the suitable dilution of secondary antibody conjugated to alkaline phosphatase (AP) or peroxidase (both from SIGMA). The elements which do not bind to the antibodies are removed and 100 μl of the substrate necessary to develop the Western are added. It is left to incubate for one hour in the dark and at 4° C. The plate is subsequently read in a plate reader with suitable wavelength and a calibration line is obtained in which the abscissa of each sample will be interpolated, which will allow the quantification of wnt-1. The results will be in μg of wnt1/carnitine.

### Example 3

### Immunohistochemical Detection of wnt-1 in Renal Biopsies

Roackland's commercial anti-human wnt-1 antibody is used as the primary antibody. The sections were mounted on a positively charged slide (Genex-brand®)

### 3.1. Deparaffinization:

Deparaffinization was achieved by means of passing the sections through xylene (10 min), and decreasing strengths of ethyl alcohol (100° 10 minutes, 96° 5 minutes, and 70° 5 minutes).

### 3.2 Blocking the Endogenous Peroxidase Activity:

The sections are incubated in 3% hydrogen peroxide solution in methanol for 15 minutes and incubated in distilled water for 10 minutes.

### 3.3 Antigen Recovery:

The sections are immersed in 10 mM citrate buffer solution pH 6, and they are heated at 121° C. in an autoclave for 15 minutes. They were left to cool for 5 minutes, and then were washed in a TBST buffer solution (50 mM Tris-HCl, 300 mM NaCl, 0.1% Tween 20, pH 7.6) bath in which they remain for 15 minutes.

### 3.4. Immunolabeling:

The tissue sections were incubated with a 1% bovine serum albumin fraction V solution (SIGMA) in TBST buffer for 5 minutes for the purpose of blocking non-specific binding sites. Then the anti-wnt-1 anti-serum is placed in a humid chamber with the suitable dilution overnight at 4° C.

### 3.5 Development of the Reaction:

The DAKO LSAB2® technique is used with AEC as a chromogenic substrate.

### 3.6 Counterstain:

The sections are immersed in Mayer's hematoxylin for 15 seconds, then they were placed under a flow of running water for development.

### 3.7 Mounting:

The mounting is performed with aqueous mount medium (VectaMount™ AQ, Vector Lab Ind)

### 3.8 Reading

The preparations are observed under a Leitz Dialux 20 EB microscope. The photographs are taken with an Olympus C4000 digital camera mounted on the microscope.

### Prophetic Example 4

### Extraction and Quantification of the WNT-1 RNA in Urine

### 4.1 Urine RNA Extraction Protocol:

At least 30 ml of fresh urine are collected and maintained in a refrigerator until the initial processing thereof (start in less than 1 hour after collection).

4.1.1 Isolation of the Cells from the Urine:

Pass the 30 ml allowed by the kit through the filter (ZRC GF™ Filter) provided by the ZR URINE RNA Isolation kit (ZYMO RESEARCH cat #R1038).

The filtered urine is discarded unless it is going to be used in another process.

### 4.1.2 RNA Extraction:

Pass 700 μL of lysate buffer of the Kit (RNA Extraction Buffer Plus™) through the column using a 1 mL syringe, collecting the cell lysate in a clean prepared RNase-free Eppendorf tube. Add to the lysate 1 volume (700 μl) of 95-100% ethanol, briefly mix well and pass the mixture through the affinity column (Zymo-Spin IC™ Column) where the RNA will be retained. Put the column on the collector tube. Centrifuge at ≧10,000 rpm for 1 minute. Remove the filtered liquid, Add 300 μl of the wash buffer (RNA Wash Buffer) to the column. Centrifuge at ≧10,000 rpm for 1 minute. Remove the filtered liquid. Add another 300 μl of the wash buffer (RNA Wash Buffer) to the column. Centrifuge at ≧10,000 rpm for 1 minute. Remove the filtered liquid. Put the column on a clean prepared RNase-free Eppendorf tube. Add 20 μL of elution buffer and wait 1 minute. Centrifuge at ≧10,000 rpm for 1 minute. Collect the filtered liquid that contains the eluted RNA. Quantify in a spectrophotometer (Nanodrop). Use immediately or store at −80° C.

### 4.2. Quantification by Means of Light-Cycler

cDNA was obtained in a final volume of 20 μl from 1 μg of total RNA using 20 pmol of oligo dTs as primers, with 100 U of the reverse transcription enzyme SuperScript II RNase H-, and 40 U of ribonuclease inhibitor (INVITROGEN), according to the supplier's instructions.

For quantification by means of Light-cycler the reactions were performed in a final volume of 20 μl, in which 0.2 μl Universal ProbeLibrary num 13 (Roche applied Science), 8.8 μl water (PCR grade), primers: left: cacctcctggccttctcc (SEQ ID NO: 4) and right: ggggcaggtacatggtgt (SEQ ID NO: 5), 4 μl Master Mix and finally 5 μl of the cDNA previously obtained are added (all the reagents are from Roche Applied Science). The TM used will be 59° C.

1-2. (canceled)

3. Method for the prognosis of chronic allograft nephropathy in patients with transplanted kidney comprising quantifying the WNT1 biomarker or a fragment thereof in a biological sample isolated from a patient;
wherein a higher level of WNT1 indicates worse prognosis than a lower level of WNT1.

4. Method according to claim 3, wherein the biological sample is urine, blood, serum or tissue biopsy and it wherein quantifying comprises determining the presence or absence of the protein, RNA or the DNA of WNT1 biomarker or a fragment thereof.

5-6. (canceled)

7. Method for the in vitro diagnosis of chronic allograft nephropathy comprising:
a) quantifying WNT1 or a fragment thereof in a biological sample isolated from a patient.
b) comparing the amount of WNT1 in the sample of step a) with the amount of WNT1 in samples isolated from healthy individuals,
wherein the presence or the relative increase of the amount of WNT1 are indicative of chronic allograft nephropathy.

8. Method according to claim 7, wherein the biological sample is a urine sample.

9. Kit for monitoring the prognosis and/or diagnosis of chronic allograft nephropathy comprising at least one molecule or composition able to bind to and recognize a sequence selected from SEQ ID No: 1, SEQ ID No: 2, SEQ ID No: 3 or a fragment thereof, optionally labeled for its detection.

10-15. (canceled)


SEQUENCE LISTING (5 sequences)

SEQ ID NO: 1 - DNA, 2368 bp, Homo sapiens
gcggtgccgcccgccgtggccgcctcagcccaccagccgggaccgcgagccatgctgtccgccgcccgcccccagggttgttaaagccagactgcgaactctcgccactgccgccaccgccgcgtcccgtcccaccgtcgcgggcaacaaccaaagtcgccgcaactgcagcacagagcgggcaaagccaggcaggccatggggctctgggcgctgttgcctggctgggtttctgctacgctgctgctggcgctggccgctctgcccgcagccctggctgccaacagcagtggccgatggtggggtattgtgaacgtagcctcctccacgaacctgcttacagactccaagagtctgcaactggtactcgagcccagtctgcagctgttgagccgcaaacagcggcgtctgatacgccaaaatccggggatcctgcacagcgtgagtggggggctgcagagtgccgtgcgcgagtgcaagtggcagttccggaatcgccgctggaactgtcccactgctccagggccccacctcttcggcaagatcgtcaaccgaggctgtcgagaaacggcgtttatcttcgctatcacctccgccggggtcacccattcggtggcgcgctcctgctcagaaggttccatcgaatcctgcacgtgtgactaccggcggcgcggccccgggggccccgactggcactgggggggctgcagcgacaacattgacttcggccgcctcttcggccgggagttcgtggactccggggagaaggggcgggacctgcgcttcctcatgaaccttcacaacaacgaggcaggccgtacgaccgtattctccgagatgcgccaggagtgcaagtgccacgggatgtccggctcatgcacggtgcgcacgtgctggatgcggctgcccacgctgcgcgccgtgggcgatgtgctgcgcgaccgcttcgacggcgcctcgcgcgtcctgtacggcaaccgcggcagcaaccgcgcttcgcgagcggagctgctgcgcctggagccggaagacccggcccacaaaccgccctccccccacgacctcgtctacttcgagaaatcgcccaacttctgcacgtacagcggacgcctgggcacagcaggcacggcagggcgcgcctgtaacagctcgtcgcccgcgctggacggctgcgagctgctctgctgcggcaggggccaccgcacgcgcacgcagcgcgtcaccgagcgctgcaactgcaccttccactggtgctgccacgtcagctgccgcaactgcacgcacacgcgcgtactgcacgagtgtctgtgaggcgctgcgcggactcgcccccaggaaacgctctcctcgagccctcccccaaacagactcgctagcactcaagacccggttattcgcccacccgagtacctccagtcacactccccgcggttcatacgcatcccatctctcccacttcctcctacctggggactcctcaaaccacttgcctggggcggcatgaaccctcttgccatcctgatggacctgccccggacctacctccctccctctccgcgggagaccccttgttgcactgccccctgcttggccaggaggtgagagaaggatgggtcccctccgccatggggtcggctcctgatggtgtcattctgcctgctccatcgcgccagcgacctctctgcctctcttcttcccctttgtcctgcgttttctccgggtcctcctaagtcccttcctattctcctgccatgggtgcagaccctgaacccacacctgggcatcagggcctttctcctccccacctgtagctgaagcaggaggttacagggcaaaagggcagctgtgatgatgtggaaatgaggttgggggaaccagcagaaatgcccccattctcccagtctctgtcgtggagccattgaacagctgtgagccatgcctccctgggccacctcctaccccttcctgtcctgcctcctcatcagtgtgtaaataatttgcactgaaacgtggatacagagccacgagtttggatgttgtaaataaaactatttattgtgctgggtcccagcctggtttgcaaagaccacctccaacccaacccaatccctctccactcttctctcctttctccctgcagccttttctggtccctcttctctcctcagtttctcaaagatgcgtttgcctcctggaatcagtatttccttccactgtagctattagcggctcctcgcccccaccagtgtagcatcttcctctgcagaataaaatctctatttttatcgatgacttggtggcttttccttgaatccagaacacaaccttgtttgtggtgtcccctatcctccccttttaccactcccag

SEQ ID NO: 2 - RNA, 2368 nt, Homo sapiens
gcggugccgcccgccguggccgccucagcccaccagccgggaccgcgagccaugcuguccgccgcccgcccccaggguuguuaaagccagacugcgaacucucgccacugccgccaccgccgcgucccgucccaccgucgcgggcaacaaccaaagucgccgcaacugcagcacagagcgggcaaagccaggcaggccauggggcucugggcgcuguugccuggcuggguuucugcuacgcugcugcuggcgcuggccgcucugcccgcagcccuggcugccaacagcaguggccgauggugggguauugugaacguagccuccuccacgaaccugcuuacagacuccaagagucugcaacugguacucgagcccagucugcagcuguugagccgcaaacagcggcgucugauacgccaaaauccggggauccugcacagcgugaguggggggcugcagagugccgugcgcgagugcaaguggcaguuccggaaucgccgcuggaacugucccacugcuccagggccccaccucuucggcaagaucgucaaccgaggcugucgagaaacggcguuuaucuucgcuaucaccuccgccggggucacccauucgguggcgcgcuccugcucagaagguuccaucgaauccugcacgugugacuaccggcggcgcggccccgggggccccgacuggcacugggggggcugcagcgacaacauugacuucggccgccucuucggccgggaguucguggacuccggggagaaggggcgggaccugcgcuuccucaugaaccuucacaacaacgaggcaggccguacgaccguauucuccgagaugcgccaggagugcaagugccacgggauguccggcucaugcacggugcgcacgugcuggaugcggcugcccacgcugcgcgccgugggcgaugugcugcgcgaccgcuucgacggcgccucgcgcguccuguacggcaaccgcggcagcaaccgcgcuucgcgagcggagcugcugcgccuggagccggaagacccggcccacaaaccgcccuccccccacgaccucgucuacuucgagaaaucgcccaacuucugcacguacagcggacgccugggcacagcaggcacggcagggcgcgccuguaacagcucgucgcccgcgcuggacggcugcgagcugcucugcugcggcaggggccaccgcacgcgcacgcagcgcgucaccgagcgcugcaacugcaccuuccacuggugcugccacgucagcugccgcaacugcacgcacacgcgcguacugcacgagugucugugaggcgcugcgcggacucgcccccaggaaacgcucuccucgagcccucccccaaacagacucgcuagcacucaagacccgguuauucgcccacccgaguaccuccagucacacuccccgcgguucauacgcaucccaucucucccacuuccuccuaccuggggacuccucaaaccacuugccuggggcggcaugaacccucuugccauccugauggaccugccccggaccuaccucccucccucuccgcgggagaccccuuguugcacugcccccugcuuggccaggaggugagagaaggauggguccccuccgccauggggucggcuccugauggugucauucugccugcuccaucgcgccagcgaccucucugccucucuucuuccccuuuguccugcguuuucuccggguccuccuaagucccuuccuauucuccugccaugggugcagacccugaacccacaccugggcaucagggccuuucuccuccccaccuguagcugaagcaggagguuacagggcaaaagggcagcugugaugauguggaaaugagguugggggaaccagcagaaaugcccccauucucccagucucugucguggagccauugaacagcugugagccaugccucccugggccaccuccuaccccuuccuguccugccuccucaucaguguguaaauaauuugcacugaaacguggauacagagccacgaguuuggauguuguaaauaaaacuauuuauugugcugggucccagccugguuugcaaagaccaccuccaacccaacccaaucccucuccacucuucucuccuuucucccugcagccuuuucuggucccucuucucuccucaguuucucaaagaugcguuugccuccuggaaucaguauuuccuuccacuguagcuauuagcggcuccucgcccccaccaguguagcaucuuccucugcagaauaaaaucucuauuuuuaucgaugacuugguggcuuuuccuugaauccagaacacaaccuuguuugugguguccccuauccuccccuuuuaccacucccag

SEQ ID NO: 3 - protein, 370 aa, Homo sapiens
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 4 - DNA, 18 bp, Artificial Sequence
  PCR Primer
cacctcctggccttctcc

SEQ ID NO: 5 - DNA, 18 bp, Artificial Sequence
  PCR Primer
ggggcaggtacatggtgt